(19) United States
(12) Patent Application Publication
(10) Pub. No.: US 2005/0196365 A1
(43) Pub. Date: Sep. 8, 2005

(54) Method for improving sensory characteristics of semisolid predominantly anhydrous lipids

(76) Inventor: Hans R. Suess, Starrkirch-Wil (CH)

Correspondence Address: Maryellen Feehery, Esq., 2500 One Liberty Place, 1650 Market Street, Philadelphia, PA, 19103, US

(21) Appl. No.: 11/099,902

(22) Filed: Apr. 5, 2005

Related U.S. Application Data
(63) Continuation-in-part of application No. 10/163,523, filed on Jun. 6, 2002.

(51) Int. Cl. A61K 7/42; A61K 7/06; A61K 7/11

(52) U.S. Cl. 424/59; 424/70.11

(57) ABSTRACT

A composition comprises amphiphobic halocarbon particles, and amphiphilic lipids having at least 2 hydroxyl groups. This composition may be used in methods of decreasing oiliness, greasiness, stickiness and tackiness of anhydrous semisolid lipids, methods of delivering pharmaceutical active ingredients, methods of preventing or treating sunburn or ultraviolet radiation, methods of preventing or treating skin disorders and methods of delivering cosmetics.

### RELATED APPLICATIONS

This application is a continuation-in-part of Ser. No. 10/163,523 filed Jun. 6, 2002 herein incorporated by reference.

### BACKGROUND OF THE INVENTION

The present invention is directed to a method of improving the adhesion of semisolid predominantly anhydrous lipids to certain substrates. This method decreases oiliness, greasiness, stickiness, and tackiness and improves the adhesion of the lipids to a substrate. These lipids may be applied to surfaces, including, but not limited to hair, leather, paper, skin, textiles and wood, where an impermeableness against water, but not water vapor, is desirable.

The principal function of the skin is to keep water in and exogenous substances out. This is accomplished by a highly ordered barrier, located in the stratum corneum, the skin's outermost layer. The stratum corneum composed of corneocytes (or horny cells) embedded in lipid lamellae, composed mainly of ceramides, cholesterol and free fatty acids. The horny layer is incessantly exposed to an oxidative environment (including, but not limited to UV radiation, ozone, aerobic microorganisms, and metallic microparticles). However, the horny layer is defended by antioxidant tocopherols, which are contained in the sebum that is continually secreted by the sebaceous glands, and ascorbic acid, which is secreted by the eccrine glands.

By frequent washing, especially of the hands, sebum and eccrine fluid is withdrawn leaving the skin exposed to increased oxidative stress, which damages a linoleic acid containing ceramide. This may result in a decreased barrier function and therefore an increased transepidermal water loss, which may ultimately cause dry skin.

Anhydrous lipid-rich preparations such as petrolatum or lanolin, which are not easily washed-off, seem appealing but they are not used since they are lacking in cosmetic elegance. Therefore, less greasy skin creams are often applied. Such skin creams are often emulsions, containing emulsifiers which are known to be potential skin irritants. Additionally, such emulsions contain a water phase which has to be preserved in order to avoid microbial contamination. Perfumes are often included in these formulations. Both the preservatives and perfumes frequently cause allergic reactions.

In the formulation of dermatological vehicles, therapeutic efficacy is an important goal. Skin penetration of active ingredients is frequently more enhanced by anhydrous semisolid excipients than by creams (which are semisolid emulsions). Anhydrous semi-solid excipients based on hydrocarbons moisturize the skin by occlusion and enhance overall skin quality more readily than creams. Furthermore, anhydrous semi-solid hydrocarbon excipients do not degrade from bacterial lipases. In spite of these advantages, creams are very often preferred to ointments, in the art, because they are more pleasant to use and therefore better accepted by patients.

On the other hand there is a disadvantage of semisolid anhydrous ointments as moisturizers. If they are applied immediately prior to dressing, and only a short time is available for absorption, then a noteworthy excess residue of the applied product is left to be absorbed by clothes, possibly affecting the actual use and acceptability.

Prior patents provide no teaching concerning the use of combinations of amphiphobic submicro or microparticles of halocarbons and amphiphilic diols, triols or polyols as effective in improving the cosmetic elegance of semisolid lipids. An amphiphilic product includes both hydrophilic and lipophilic groups. An amphiphobic product does not include either a hydrophilic or a lipophilic group.

Other patents in this field include British Patent 1575 201 (filed Oct. 22, 1975); U.S. Pat. No. 4,087,517 (1976); Japanese Patent No. 6228 3920; European Patent No. 0 598 412; and WO 02/03946 A1.

The greasier and stickier a moisturizer is, the less cosmetically acceptable it is. Increases in the lipid content of a moisturizer often increase the greasiness and stickiness of the moisturizer. For this reason, it is desirable to suppress the greasiness and stickiness of lipid-rich preparations (e.g. petrolatum or other formulations consisting mainly of hydrocarbons). As was recognized twenty years ago, “A boon to all sufferers from dry skin would be a product with the efficacy of petrolatum and the hedonic-aesthetic properties of a light oil in water cream.” (Kligman A. M. and Leyden J. J.: Safety and Efficacy of Topical Drugs and Cosmetics, New York Grune & Stratton, 1982, p. 236.)

Water is the plasticiser of the skin. Lipid content is of major importance to the plasticity of the skin because the presence of lipids decreases transepidermal water loss and consequently increases moisture content of the stratum corneum. Therefore, lipid-rich formulations are effective in the treatment of dry skin.

### SUMMARY OF THE INVENTION

The present invention is quite unexpected. The sensory characteristics, the aesthetic appeal and thereby the user acceptance of anhydrous semisolid lipids can be greatly improved with the addition of a combination of amphiphobic halocarbon particles (preferably in one embodiment micro or sub-micro particles of polymerized perhalogenoalkenes) and at least one amphiphilic lipid containing at least two hydroxyl groups. The amphiphilic lipid is substantially insoluble in water and substantially miscible with the anhydrous semi-solid lipid.

Examples of such semisolid lipids are mainly complex mixtures of hydrocarbons (e.g., without limitation, petrolatum) or combinations of liquid hydrocarbons (e.g., without limitation, paraffin oils or oligomers of monoolefins, e.g., without limitation, ethylene, propylene, butylene, isobutylene, and decene) with solid polymers of monoolefins, esters and combinations of liquid and/or solid esters (e.g., without limitation, waxes), semisoft triglycerides or any combination thereof (e.g., without limitation, lanolin or wool wax).

The lipids, used in this invention, contain minimal or no quantities of water, (referred to as “anhydrous”), preferably no water. The amount depends on the chemical composition. For example, centrifuged raw wool wax contains 1-5% water. At 32° C. (the approximate temperature of the skin surface), water is only very slightly soluble in liquid hydrocarbons (less than 75 ppm), whereas esters (e.g., without limitation, dibutyladipat) may contain up to 100 times more water.

Lipids with high water content are less occlusive than lipids with low water content. Hydrocarbons, which have a low water content, excel in the treatment of dry skin by occlusion.

Branched lipids (e.g., without limitation, petrolatum) are generally sticky and greasy. Additionally, bulky lipids (e.g., without limitation, anhydrous lanolin) also tend to be sticky and greasy. On the other hand, straight waxes (e.g., without limitation, jojoba oil or mixtures with other waxes, e.g., without limitation, beeswax, carnauba, or candelilla wax) are much less greasy and sticky. However, patient compliance may be improved by the present invention because the skin feel is further improved.

Suitable halocarbon microparticles have diameters in the range of 0.1 (or smaller) to 50 micrometers, such as Algoflon HC® from Ausimont, and Fluortress from DuPont. See, International Cosmetic Ingredients Dictionary and Handbook, 8th Ed 2000.

The dry powders of the amphiphobic halocarbon particles may be agglomerated. In which case, the particles have to be de-agglomerated in order to obtain an even distribution of the particles in the final composition. This can be accomplished by using volatile solvents which show some affinity to the halopolymer. Preferred solvents include, but are not limited to, volatile silicones, linear or cyclic, having from 2 to 7 silicon atoms and from 1 to 10 alkyl groups (e.g., without limitation, hexamethyldisiloxane, dimethicones, octamethylcyclotetrasiloxane, decamethylcyclopenta-siloxane, dimethicone siloxane, octamethyltrisiloxane and/or mixtures thereof). Perfluorobutyl ether and methylperfluoroisobutylether (e.g., without limitation, HFE-7100 and HFE-7200 from 3M Chemicals), which show a high affinity to the halogenpolymers, are preferred solvents. Other preferred solvents are C₈₋₉ to C₁₂₋₂₀ isoparaffins (e.g., without limitation, Isopar® from Exxon Corporation). The solvents may be used individually or in combination.

The submicro or micro particles of the perhalogenopolymers may be dispersed in a convenient liquid. Micronized or submicronized perhalopolymers have been known in the art for more than a quarter of a century. However, until the present invention, they did not work as expected in the field of skin protectants and skin care because semisolid anhydrous preparations containing such particles lack cosmetic elegance. Therefore, the compliance of treatments with such preparations was unsatisfactory. Additionally, when the micronized or submicronized particles are used as a component of an emulsion, they lose a great part of their sensorial qualities (often from interactions with emulsifiers).

The prior art then incorporates the halocarbon microparticles in an anhydrous vehicle such as described in U.S. Pat. No. 4,355,046 (Süess 1982). Such vehicles are based on petrolatum and microcrystalline wax (ozokerite) and volatile silicones like hexamethyldisiloxane, octamethylcyclotetrasiloxane or decamethylpentasiloxane. But the addition of micronized polytetrafluoroethylene did not bring the expected results regarding cosmetic elegance.

In one embodiment of this invention, the amphiphilic lipid is phytantriol (3,7,11,15-tetramethyl-1,2,3-trihydroxy-hexadecane) which is a strong hydrophobic emollient, cutting down the water loss from the skin (moisturizer) unexpectedly delivered a vehicle with supreme hedonic properties regarding skin feel and skinprotecting properites.

Phytantriol has three hydroxyl groups on one side and a long saturated hydrocarbon chain on the other side of the molecule. Phytantriol acts as moisturizer and a penetration enhancer and acts as a spreading agent for UV filters.

In one embodiment, the solubility parameters of cyclomethicones, e.g., without limitation, white mineral oil and petrolatum, are in the 5.77 to 7.33 range, whereas that of propylene glycol is 14.0 and glycerol is 16.26. A difference of smaller than 2 usually indicates mutual solubility but polar forces and hydrogen bonding are known to greatly affect this span. (Vaughan C. D., J. Soc. Cosmet. Chem. 36, 319-333, 1985).

Examples of suitable diols/polyols in accordance with the present invention for improving the sensory properties of semisolid embodiments are lipid-soluble molecules containing 2 or more hydroxyls at one side of the molecule and an (preferably unsubstituted) hydrocarbon chain of at least 8 C-atoms at the other side (a composition similar to phytantriol) including, but not limited to, phytantriol, certain ceramides (6), sphinganine, esters or ethers of diols/triols, including but not limited to, glyceryl laurate, glyceryl isostearate, glyceryl stearate, glyceryl oleate or of other polyols, which contain more than three hydroxyl groups like sorbitol, erythritol without limitation (all containing a hydrocarbon chain of at least 8 C-atoms).

### DETAILED DESCRIPTION OF THE INVENTION

Compositions of the present invention with their superior skin feel are ideal excipients for dermatological products since they adhere well to skin, and are not easily washed off. For these reasons, active ingredients, delivered by these excipients, remain in contact with the skin longer. In most cases, an increased occlusivity induces a higher moisturization of the skin, and as a consequence, an increased transdermal absorption of active ingredients. In some embodiments, varying the type and amount of amphiphilic bis/oligo hydroxylated lipids, the occlusive effect can be varied correspondingly.

Many formulations have been made in laboratory experiments to show the benefits of the various embodiments of the invention and the ingredients used.

### EXAMPLE 1

| Weight % | |
|---|---|
| 45 | Petrolatum USP |
| 20 | Ozokerite |
| 2 | Phytantriol |
| 2 | PTFE |
| 31 | Volatile Silicone |

Heat petrolatum and ozokerite in a container equipped with a stirrer to 95° C. When an a homogenous liquid is obtained, add PTFE and phytantriol dispersed in a second container equipped with a homogenizer at 40° C. Maintain 40° C. while continually stirring.

The concluding assement of an investigation of the protective effect of this preparation by means of the repeated wash test procedure at the Municipal Clinique of Dermatolgoy in Karlsruhe (Clinical director W. Gehring) says: “While at the same time being extremely well accepted from a cosmetic point of view this lipogel has shown itself, using the repeated wash test method, to be effective at counteracting the damaging effects of sodium lauryl sulfate. A loss in epidermal moisture was able to be avoided and the rise in TEWL was able to be reduced considerably. As far as TEWL is concerned, petrolatum also had a similar effect. Due to its poor cosmetic acceptance, however, it can hardly be considered as a preparation for protecting the skin. The new lipogel is a non-occlusive skin protective that, from a cosmetic point of view, is extremely well tolerated. It counteracts washing agents or surfactants by moistening the skin and stabilizing the function of the epidermal barrier.”

### EXAMPLE 2

| | Weight % |
|---|---|
| Petrolatum | 45 |
| Ozokerite | 17.5 |
| Glyceryl Stearate (mono) | 2 |
| PTFE | 2 |
| Volatile Silicone | 33.45 |

Petrolatum, ozokerite and glyceryl stearate are heated together until 95° C., then the procedure in Example 1 is followed.

This preparation has cosmetic properties close to Example 1. If glyceryl laurate is used instead of glyceryl stearate then the cosmetic acceptance is diminished (probably because it has a higher solubility parameter (9.08 vs. 8.31 of glyceryl steartate, petrolatum 7.33 and volatile silicon 5.99).

### EXAMPLE 3

| | Weight % |
|---|---|
| Polydecene (preferably Nexbase FG, Neste) | 70 |
| Polythene (preferably Luwax A, BASF) | 11 |
| Phytantriol | 1.8 |
| PTFE | 2.2 |
| Volatile Silicone (preferably Dow Corning 2-1184) | 15 |

Heat polydecene and Polythene while stirring to 115° C., then cool to 80° C. add Phytantriol, and then add the PTFE dispersed in the volatile silicone while stirring and cool to 40° C.

It is understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims.

1. A method of hydrating and protecting skin by forming a non-tacky layer on the skin comprising applying a composition to the skin wherein the composition comprises at least one anhydrous semisolid lipid, at least one amphiphobic halocarbon particles and at least one amphiphilic lipid having at least 2 hydroxyl groups, wherein the composition is anhydrous and the amphiphilic lipid is substantially insoluble in water and substantially miscible with the anhydrous semi-solid lipid.

2. The method of claim 1 wherein the amphiphobic halocarbon particles are selected from the group consisting of polytetrafluoroethylene, polychlorotrifluoroethylene, polyhexafluoropropylene, polyvinylidine fluoride, polyvinylfluoride, copolymer mixtures of tetrafluoroethylene and ethylene, and mixtures of tetrafluoroethylene, propylene and fluorinated copolymers of ethylene propylene.

3. The method of claim 2 wherein the amphiphobic halocarbon particles are present at about 0.2% to about 20%.

4. The method of claim 2 wherein the amphiphobic halocarbon particles are present at about 0.5% to about 5%.

5. The method of claim 2 wherein the amphiphobic halocarbon particles are present at about 1.5% to about 2.5%.

6. The method of claim 1 wherein the amphiphilic lipids are selected from the group consisting of diols, triols and polyols.

7. The method of claim 6 wherein the amphiphilic lipids are miscible in the anhydrous semi-solid lipid and have a molecular weight of less than 1000.

8. The method of claim 1 wherein the amphiphobic halocarbon particles are dispersed in a liquid, the liquid comprises a volatile liquid.

9. The method of claim 8 wherein the volatile liquid has a boiling point below 250° C.

10. The method of claim 8 wherein the volatile liquid is selected from the group consisting of hexamethyldisiloxane, octamethylcyclotetrasiloxane, decamethylcyclopentasiloxane, octamethyltrisiloxane, dimethicone and mixtures thereof.

11. The method of claim 8 wherein the volatile liquid is selected from the group consisting of C₁₁₋₁₃ isoparaffin.

12. The method of claim 11 wherein the paraffin is present at about 0% to about 60%.

13. A method of delivering pharmaceutical active ingredients comprising combining at least one anhydrous semisolid lipid, at least one amphiphobic halocarbon particles and at least one amphiphilic lipid having at least 2 hydroxyl groups and adding at least one pharmaceutical active ingredient to the combination, wherein the combination is anhydrous and the amphiphilic lipid is substantially insoluble in water and substantially miscible with the anhydrous semi-solid lipid.

14. A method of preventing or treating sunburn or ultraviolet radiation comprising topically applying a composition of at least one anhydrous semisolid lipid, one or more sunscreens, amphiphobic halocarbon particles and at least one amphiphilic lipid having at least 2 hydroxyl groups, wherein the composition is anhydrous and the amphiphilic lipid is substantially insoluble in water and substantially miscible with the anhydrous semi-solid lipid.

15. A method of preventing or treating skin disorders comprising topically applying a composition comprising at least one anhydrous semisolid lipid, at least amphiphobic halocarbon particles and at least one amphiphilic lipid having at least 2 hydroxyl groups, wherein the composition is anhydrous and the amphiphilic lipid is substantially insoluble in water and substantially miscible with the anhydrous semi-solid lipid.

16. The method of claim 15 wherein the skin disorder is dry skin.

17. A method of delivering cosmetics to skin comprising topically applying a combination of at least one anhydrous semisolid lipid, one or more cosmetics, at least one amphiphobic halocarbon particles and at least one amphiphilic lipid having at least 2 hydroxyl groups, wherein the combination is anhydrous and the amphiphilic lipid is substantially insoluble in water and substantially miscible with the anhydrous semi-solid lipid.

18. A composition comprising at least one anhydrous semisolid lipid, at least one amphiphobic halocarbon particles; and at least one amphiphilic lipids having at least 2 hydroxyl groups, wherein the composition is anhydrous and the amphiphilic lipid is substantially insoluble in water and substantially miscible with the anhydrous semi-solid lipid.

19. The composition of claim 18 further comprising one or more pharmaceutically active ingredients.

20. The composition of claim 18 further comprising one or more sunscreens.

21. The composition of claim 18 further comprising one or more cosmetic ingredients.

22. The composition of claim 18 wherein the amphiphobic halocarbon particles are selected from the group consisting of polytetrafluoroethylene (PTFE), polychlorotrifluoroethylene, polyhexafluoropropylene, polyvinylidine fluoride, polyvinylfluoride, copolymer mixtures of tetrafluoroethylene and ethylene, and mixtures of tetrafluoroethylene, propylene and fluorinated copolymers of ethylene propylene.

23. The composition of claim 22 wherein the amphiphobic halocarbon particles are present at about 0.2% to about 20%.

24. The composition of claim 22 wherein the amphiphobic halocarbon particles are present at about 0.5% to about 5%.

25. The composition of claim 22 wherein the amphiphobic halocarbon particles are present at about 1.5% to about 2.5%.

26. The composition of claim 18 wherein the amphiphilic lipids are selected from the group consisting of diols, triols and polyols.

27. The composition of claim 26 wherein the amphiphilic lipids are miscible in the anhydrous semi-solid lipid and have a molecular weight of less than 1000.

28. The composition of claim 18 wherein the amphiphobic halocarbon particles are dispersed in a liquid, the liquid comprises a volatile liquid.

29. The composition of claim 28 wherein the volatile liquid has a boiling point below 250° C.

30. The composition of claim 28 wherein the volatile liquid is selected from the group consisting of hexamethyldisiloxane, octamethylcyclotetrasiloxane, decamethylcyclopentasiloxane, octamethyltrisiloxane, dimethicone and mixtures thereof.

31. The composition of claim 28 wherein the volatile liquid is selected from the group consisting of C₁₁₋₁₃ isoparaffin.

32. The composition of claim 31 wherein the paraffin is present at about 0% to about 60%.
